(19) United States
(12) Patent Application Publication
(10) Pub. No.: US 2012/0245193 A1
(43) Pub. Date: Sep. 27, 2012

(54) PERIPHERIALLY ACTING μ OPIOID ANTAGONISTS

(75) Inventors: Bernard Silverman, Needham, MA (US); Mark Todtenkopf, Franklin, MA (US); Daniel Deaver, Franklin, MA (US); Lauren DiPetrillo, Somerville, MA (US)

(73) Assignee: Alkermes, Inc.

(21) Appl. No.: 13/396,152

(22) Filed: Feb. 14, 2012

Related U.S. Application Data
(60) Provisional application No. 61/442,629, filed on Feb. 14, 2011.

(51) Int. Cl. A61K 31/485; C07D 221/28; C07D 489/08; A61P 1/00

(52) U.S. Cl. 514/282; 514/289; 546/74; 546/45

(57) ABSTRACT

The present invention relates to a novel functional peripherally-acting μ opioid receptor antagonist of Formula I:

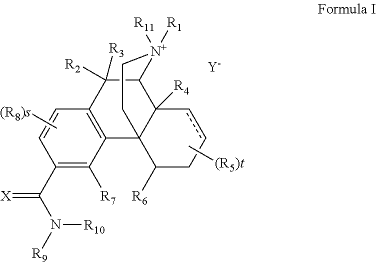

The novel compounds of the present invention have reduced oral bioavailability without compromising opiate-induced analgesia in the CNS. The compounds of the present invention are further efficacious at low doses and are useful in treating gastrointestinal conditions associated with opioid analgesic therapy.

### RELATED APPLICATION(S)

This application claims the benefit of U.S. Provisional Application No. 61/442,629, filed Feb. 14, 2011. The entire teaching of the above application is incorporated herein by reference.

### TECHNICAL FIELD

This invention relates to a peripherally-acting μ opioid receptor antagonist having reduced oral bioavailability and a high therapeutic window useful in treating gastrointestinal conditions associated with opioid analgesic therapy.

### BACKGROUND OF THE INVENTION

Opioids are the most commonly used pharmacotherapy to treat patients with moderate to severe pain. Despite many attempts to develop alternate therapeutics, opioid analgesics remain the mainstay of therapy in many patients suffering from moderate to severe pain. Unfortunately, the use of opioid analgesics is associated with a number of adverse effects among which those affecting the gastrointestinal (GI) tract are the most troublesome in terms of frequency and severity. Constipation is the most common adverse affect associated with the use of opioid analgesics. However, opioid therapy also affects bowel function by causing opioid induced bowel dysfunction. This condition is characterized by accumulation of gas and secretions, and retention of bowel content, leading to hard stool, incomplete evacuation, bloating, pain, nausea and vomiting (Pappagallo M. Incidence, prevalence and management of opioid bowel dysfunction. Am J Surg, 2001; 182 (suppl 5a):11s-18s; Kurz A, Sesser I. Opiate-induced bowel dysfunction: pathophysiology and potential new therapies. Drugs 2003; 63:649-71).

The pharmacologic management of treating adverse effects associated with long term opioid use (e.g., in the control of chronic pain) involves two approaches: non-specific with laxatives and specific treatment with opioid receptor antagonists. Although laxative treatment is usually the first recommendation that physicians make for the treatment of constipation, it does not address the underlying cause of opioid receptor mediated constipation. Due to limited efficacy, frequency of dose adjustments, combination therapy and laxative switching, only about 50% of patients experience satisfactory treatment with laxatives (Holzer, P. Opioid receptors in the gastrointestinal tract. Regulatory peptides, 2009; 155:11-17).

Newer targeted treatment with opioid receptor antagonists is also prone to issues. Naloxone, a μ opioid antagonist, blocks opioid action at the level of the intestinal receptor and has low systemic bioavailability due to a marked hepatic first pass effect. Several clinical studies have shown that oral naloxone is able to improve opiate induced bowel dysfunction without compromising opiate-induced analgesia. However, naloxone can easily to cross the blood brain barrier and hence reverse analgesia if given at sufficient doses (Kurz A, Sesser I. Opiate-induced bowel dysfunction: pathophysiology and potential new therapies. Drugs, 2003; 63:649-71). Therefore the therapeutic range of immediate release naloxone is narrow because of the need to titrate peripherally versus centrally active doses (Sykes N P. An investigation of the ability of oral naloxone to correct opioid related constipation in patients with advanced cancer. Palliat Med, (1996) 10:135-144).

Methylnaltrexone (available as RELISTOR®) is an option for treating opioid-induced constipation in people receiving palliative care who have not responded to adequately titrated laxatives. Methylnaltrexone is a quaternary derivative of naltrexone that does not cross the blood brain barrier and acts as a selective peripheral opioid receptor antagonist. Methylnaltrexone is delivered via subcutaneous injection and studies have shown that it effectively prevents morphine induced changes in gastro-intestinal motility and transit without affecting analgesia (De Schepper H U, Cremonini F, Park M I, Camilleri M. Opioid and the gut:pharmacology and current clinical experience. Neurogastroenterol Motil, (2004) 16: 383-394). Further characterization and validation with controlled clinical trials are awaited to expand the use of methylnaltrexone in patients with opioid induced bowel dysfunction who are not receiving palliative care.

Alvimopan, a selective peripherally acting μ receptor antagonist has been shown to reduce opioid-induced bowel symptoms without antagonizing centrally mediated opioid effects. Compared to methylnaltrexone, in-vitro data show that alvimopan has a higher binding affinity for human μ opioid receptors and is more portent (Becker G, Blum H E. Novel opioid antagonists for opioid-induced bowel dysfunction and postoperative ileus. Lancet, (2009) 373:1198-1206). Clinical trials have further demonstrated that the proportion of gastrointestinal related adverse events was lower in patients treated with Alvimopan than with placebo, however, serious cardiovascular adverse events arose in patients with established or high risk of cardiovascular disease (Becker G, Blum H E. Novel opioid antagonists for opioid-induced bowel dysfunction and postoperative ileus. Lancet, (2009) 373:1198-1206). As such, Alvimopan's approval was limited in scope to inpatient use for the management of post operative ileus.

Because the analgesic properties of opioid agonists are mediated primarily by their effects in the central nervous system (CNS) (Russell J, Bass P, Goldberg L I, Schuster C R, Merz H. Antagonism of gut, but not central effects of morphine with quaternary narcotic antagonists. Eur J Pharmacol, (1982) 78: 255-261), and side effects are mediated primarily by their actions in the peripheral nervous system (Moss J, Rosow C E. Development of peripheral opioid antagonist: new insights into opioid effects. (2008) 1116-1130), a potential strategy to reduce these side effects is to treat with an opioid antagonist that has its effects primarily in the enteric nervous system (ENS). As such, there is an immediate need to discover novel peripherally acting opioid modulators to treat gastrointestinal conditions associated with opioid analgesic therapy.

### SUMMARY OF THE INVENTION

The present invention relates to the unexpected discovery that Compound 1, a peripherally restricted opioid antagonist that is functionally selective for μ opioid receptors, with little-to-no activity at δ or κ receptors is an efficacious for the treatment and prevention of opioid-induced bowel dysfunction and other gastrointestinal conditions associated with opioid analgesic therapy.

The present invention relates to a method of alleviating the adverse conditions associated with opioid analgesic therapy by the oral administration of a compound of Formula I:

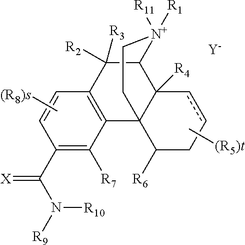

wherein:
s is 0, 1 or 2;
t is 0, 1, 2, 3, 4, 5, 6, or 7;
Y— is a pharmaceutically acceptable counterion;

### X is S or O;

each R₂, R₃, R₄, R₅, R₆, R₇ and R₈ is independently selected from absent, hydrogen, halogen, —OR₂₀, —SR₂₀, —NR₂₀R₂₁, —C(O)R₂₀, —C(O)OR₂₀, —C(O)NR₂OR₂₁, —N(R₂₀)C(O)R₂₁, —CF₃, —CN, —NO₂, —N₃, acyl, alkoxy, substituted alkoxy, alkylamino, substituted alkylamino, dialkylamino, substituted dialkylamino, substituted or unsubstituted alkylthio, substituted or unsubstituted alkylsulfonyl, optionally substituted aliphatic, optionally substituted aryl, heterocyclyl or substituted heterocyclyl; or alternatively, two of R₂, R₃, R₄, R₅, R₆, R₇ and R₈ together with the atoms they are attached to form an optionally substituted ring; alternatively R₂ and R₃ together with the carbon they are attached to form a C═X group; wherein each R₂₀ and R₂₁ is independently selected from absent, hydrogen, halogen, —OH, —SH, —NH₂, —CF₃, —CN, —NO₂, —N₃, —C(O)OH, —C(O)NH₂, acyl, alkoxy, substituted alkoxy, alkylamino, substituted alkylamino, dialkylamino, substituted dialkylamino, substituted or unsubstituted alkylthio, substituted or unsubstituted alkylsulfonyl, aliphatic, substituted aliphatic, aryl or substituted aryl; and
each R₁ and R₁₁ is selected from —C(O)OR₂₀, —C(O)NR₂OR₂₁, —CF₃, acyl, alkoxy, substituted alkoxy, alkylamino, substituted alkylamino, dialkylamino, substituted dialkylamino, alkylthio, substituted alkylthio, alkylsulfonyl, substituted alkylsulfonyl, aliphatic, substituted aliphatic, aryl, substituted aryl, heterocyclyl or substituted heterocyclyl;
alternatively two of R₁, R₉, R₁₀ and R₁₁ together with the atoms they are attached to form an optionally substituted ring; alternatively two R₅ groups, or an R₅ and an R₆ group, together with the carbon they are attached to form a C═X group;
each R₉ and R₁₀ is selected from hydrogen, aliphatic, substituted aliphatic, aryl, substituted aryl, heterocyclyl or substituted heterocyclyl.

In particular the invention relates to a method wherein, said compound of Formula I is administered in a daily dosage of about 1 to about 300 mg/day, preferably about 2 to about 150 mg/day, preferably about 5 to about 100 mg/day, or preferably about 5 to about 50 mg/day.

The present invention further relates to methods of alleviating the adverse conditions associated with opioid analgesic therapy by the oral administration of a compound wherein said compound has the following Formula II:

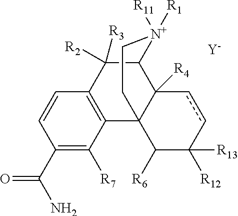

wherein;
t is 0, 1, 2, 3, 4, 5, 6, or 7;
Y— is a pharmaceutically acceptable counterion;
each R₂ and R₃ is independently selected from hydrogen, halogen, —OR₂₀, and —SR₂₀;
alternatively R₂ and R₃ together with the carbon they are attached to form a C═X group;
each R₄, R₆, and R₇ is independently selected from absent, hydrogen, halogen, —OR₂₀, —SR₂₀, —NR₂OR₂₁, —C(O)R₂₀, —C(O)OR₂₀, —C(O)NR₂OR₂₁, —N(R₂₀)C(O)R₂₁, —CF₃, —CN, —NO₂, —N₃, acyl, alkoxy, substituted alkoxy, alkylamino, substituted alkylamino, dialkylamino, substituted dialkylamino, substituted or unsubstituted alkylthio, substituted or unsubstituted alkylsulfonyl, aliphatic, substituted aliphatic, aryl, substituted aryl, heterocyclyl or substituted heterocyclyl; alternatively, two of R₆ and R₇ together with the atoms they are attached to form an optionally substituted ring;
each R₁ and R₁₁ is selected from aliphatic, substituted aliphatic, aryl, substituted aryl, heterocyclyl or substituted heterocyclyl; and
each R₁₂ and R₁₃ is independently selected from hydrogen, halogen, —OR₂₀, and —SR₂₀;
alternatively R₂ and R₃ together with the carbon they are attached to form a C═X group.

In particular the invention relates to a method wherein, said compound of Formula II is administered in a daily dosage of about 1 to about 300 mg/day, preferably about 5 to about 150 mg/day, more preferably about 5 to about 100 mg/day, even more preferably about 5 to about 50 mg/day.

In a preferred embodiment, R₁ is an alkyl group, preferably methyl, ethyl or propyl.

In a preferred embodiment, R₇ is hydroxyl; R₆ is hydrogen; and R₁₂ and R₁₃ together form a carbonyl group.

In another aspect, said condition associated with opioid analgesic therapy is a gastrointestinal disease or disorder, including but not limited, to opioid induced bowel dysfunction, opioid related post operative ileus, constipation, incomplete evacuation, abdominal distention, bloating, abdominal discomfort, and interference with oral drug administration and absorption.

In yet another aspect the methods of the present invention treat adverse conditions associated with the administration of opioid analgesics, including but not limited to, morphine, diamorphine, fentanyl, alfentanil, buprenorphine, oxycodone, hydromorphone, methadone, codeine, tramadol and butorphanol.

In a further aspect, the present invention relates to a method of treating or preventing pain while mitigating opioid-induced constipation comprising oral administration to a patient in need of such treatment, a therapeutically effective amount of compound according to Formula I or II.

In still a further aspect, the present invention relates to a unit dosage formulation for alleviating the adverse conditions associated with opioid analgesic therapy by the oral administration of a compound according to Formula I or II.

### BRIEF DESCRIPTION OF DRAWINGS

FIG. 1: Change from baseline in spontaneous bowel movement (SBM) and complete spontaneous bowel movement in patients.

FIG. 2: Inhibition of opioid-induced constipation by oral and IP administration of methyl naltrexone (MNTX).

FIG. 3: Inhibition of opioid-induced constipation by oral and IP administration of Compound-1.

FIG. 4: Response latency in PGE₂ induced gut motility study of methyl naltrexone (MNTX) with various oral (PO) and intraperitoneal (IP) administration.

FIG. 5: Response latency in PGE₂ induced gut motility study of Compound-1 with various oral (PO) and intraperitoneal (IP) administration.

FIG. 6: Comparison of the duration of action of oral administration of Compound-1 and methyl naltrexone.

FIG. 7: Comparison of the duration of action of the minimal effective IP dose for MNTX and PO dose for Compound-1.

FIG. 8: Change from baseline in spontaneous bowel movement (SBM) and complete spontaneous bowel movement in patients.

FIG. 9: Pharmacokinetic data for Compound-1 and Compound-30 in rats.

FIG. 10: Response latency in PGE₂ induced gut motility study of Compound-2 with various oral (PO) and intraperitoneal (IP) administration.

FIG. 11: Response latency in PGE₂ induced gut motility study of Compound-3 with various oral (PO) and intraperitoneal (IP) administration.

FIG. 12: Response latency in PGE₂ induced gut motility study of Compound-4 with various oral (PO) and intraperitoneal (IP) administration.

### DETAILED DESCRIPTION OF THE INVENTION

The present invention provides a method of alleviating adverse conditions associated with opioid analgesic therapy in a subject in need thereof. The method comprises administering to a subject in need thereof a therapeutically effective amount, 1 to about 300 mg/day, preferably about 5 to about 150 mg/day, more preferably about 5 to about 100 mg/day, even more preferably about 5 to about 50 mg/day of a peripherally-acting μ opioid receptor antagonists having reduced oral bioavailability or a pharmaceutically acceptable salt, hydrate or solvate thereof. The peripherally-acting μ opioid receptor antagonist is represented by Formula I, or a pharmaceutically acceptable salt, hydrate or solvate thereof

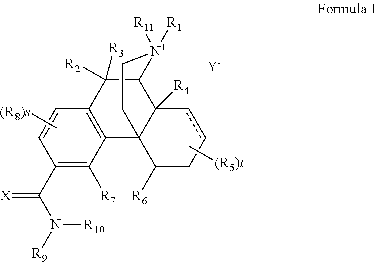

wherein:
s is 0, 1 or 2;
t is 0, 1, 2, 3, 4, 5, 6, or 7;
Y— is a pharmaceutically acceptable counterion;

### X is S or O;

each R₂, R₃, R₄, R₅, R₆, R₇ and R₈ is independently selected from absent, hydrogen, halogen, —OR₂₀, —SR₂₀, —NR₂OR₂₁, —C(O)R₂₀, —C(O)OR₂₀, —C(O)NR₂OR₂₁, —N(R₂₀)C(O)R₂₁, —CF₃, —CN, —NO₂, —N₃, acyl, alkoxy, substituted alkoxy, alkylamino, substituted alkylamino, dialkylamino, substituted dialkylamino, substituted or unsubstituted alkylthio, substituted or unsubstituted alkylsulfonyl, optionally substituted aliphatic, optionally substituted aryl, heterocyclyl or substituted heterocyclyl; or alternatively, two of R₂, R₃, R₄, R₅, R₆, R₇ and
R₈ together with the atoms they are attached to form an optionally substituted ring;
alternatively R₂ and R₃ together with the carbon they are attached to form a C═X group;
wherein each R₂₀ and R₂₁ is independently selected from absent, hydrogen, halogen, —OH, —SH, —NH₂, —CF₃, —CN, —NO₂, —N₃, —C(O)OH, —C(O)NH₂, acyl, alkoxy, substituted alkoxy, alkylamino, substituted alkylamino, dialkylamino, substituted dialkylamino, substituted or unsubstituted alkylthio, substituted or unsubstituted alkylsulfonyl, aliphatic, substituted aliphatic, aryl or substituted aryl; and
each R₁ and R₁₁ is selected from —C(O)OR₂₀, —C(O)NR₂OR₂₁, —CF₃, acyl, alkoxy, substituted alkoxy, alkylamino, substituted alkylamino, dialkylamino, substituted dialkylamino, alkylthio, substituted alkylthio, alkylsulfonyl, substituted alkylsulfonyl, aliphatic, substituted aliphatic, aryl, substituted aryl, heterocyclyl or substituted heterocyclyl;
alternatively two of R₁, R₉, R₁₀ and R₁₁ together with the atoms they are attached to form an optionally substituted ring; alternatively two R₅ groups, or an R₅ and an R₆ group, together with the carbon they are attached to form a C═X group;
each R₉ and R₁₀ is selected from hydrogen, aliphatic, substituted aliphatic, aryl, substituted aryl, heterocyclyl or substituted heterocyclyl.

In a preferred embodiment, the peripherally-acting μ opioid receptor antagonist is represented by Formula II, or a pharmaceutically acceptable salt, hydrate or solvate thereof

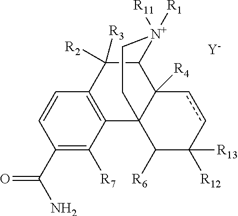

wherein;
t is 0, 1, 2, 3, 4, 5, 6, or 7;
Y— is a pharmaceutically acceptable counterion;
each R₂ and R₃ is independently selected from hydrogen, halogen, —OR₂₀, and —SR₂₀;
alternatively R₂ and R₃ together with the carbon they are attached to form a C═X group;
each R₄, R₆, and R₇ is independently selected from absent, hydrogen, halogen, —OR₂₀, —SR₂₀, —NR₂OR₂₁, —C(O)R₂₀, —C(O)OR₂₀, —C(O)NR₂OR₂₁, —N(R₂₀)C(O)R₂₁, —CF₃, —CN, —NO₂, —N₃,
acyl, alkoxy, substituted alkoxy, alkylamino, substituted alkylamino, dialkylamino, substituted dialkylamino, substituted or unsubstituted alkylthio, substituted or unsubstituted alkylsulfonyl, aliphatic, substituted aliphatic, aryl, substituted aryl, heterocyclyl or substituted heterocyclyl; alternatively, two of R₆ and R₇ together with the atoms they are attached to form an optionally substituted ring;
each R₁ and R₁₁ is selected from aliphatic, substituted aliphatic, aryl, substituted aryl, heterocyclyl or substituted heterocyclyl; and
each R₁₂ and R₁₃ is independently selected from hydrogen, halogen, —OR₂₀, and —SR₂₀;
alternatively R₂ and R₃ together with the carbon they are attached to form a C═X group.

In a more preferred embodiment, the peripherally-acting μ opioid receptor antagonist is represented by Formula II, wherein R₁ is selected from: —(CH₂)ₙ-c-C₃H₅, —(CH₂)ₙ-c-C₄H₇, —(CH₂)—ₙ-c-C₅H₉, —(CH₂)ₙ—CH═CH₂ or —(CH₂)ₙ—CH═C(CH₃)₂ wherein n is independently 0, 1, 2 or 3.

R₂ and R₃ are independently H, —OH or —SH.
R₆ and R₇ are independently H, —OH or together R₆ and R₇ form an —O— or —S— group.
R₁₂ and R₁₃ are independently H, —OH, OCH₃ or together R₆ and R₇ form a ═O or ═CH₂ group.

In a preferred embodiment, Y— is selected from hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate. In a more preferred embodiment, the counterion is selected from bromide, chloride, maleate and malate.

Representative compounds according to Formula I include the following:

TABLE A

| | |
|---|---|
| 1 | 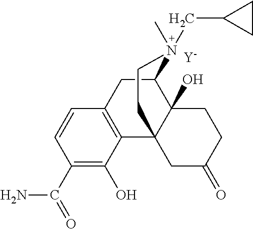 |
| 2 | 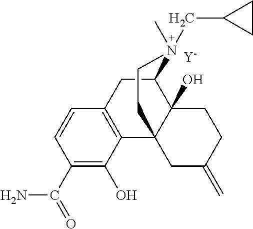 |
| 3 | 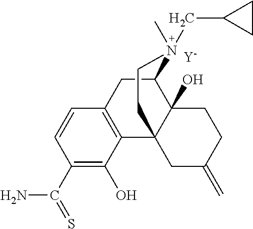 |
| 4 | 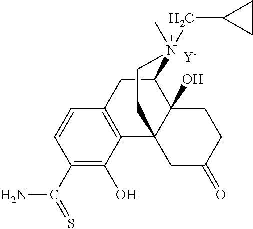 |
| 5 | 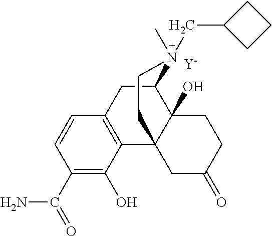 |
| 6 | 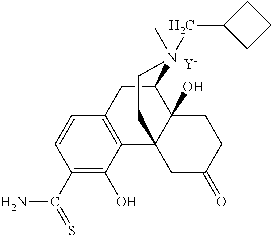 |
| 7 | 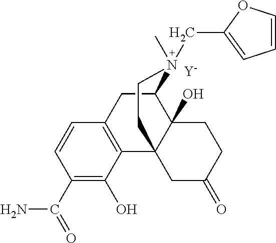 |
| 8 | 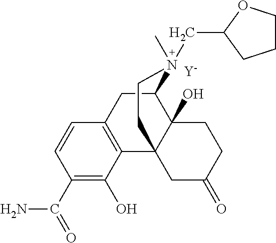 |
| 9 | 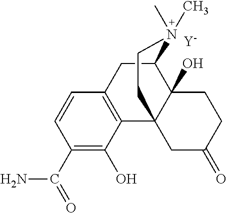 |
| 10 | 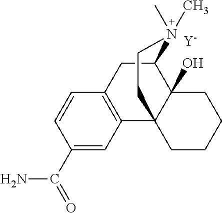 |
| 11 | 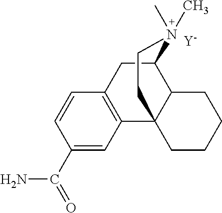 |
| 12 | 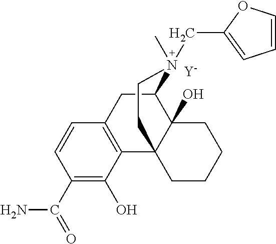 |
| 13 | 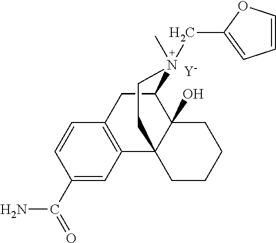 |
| 14 | 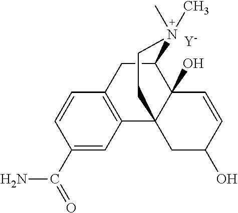 |
| 15 | 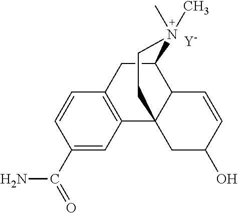 |
| 16 | 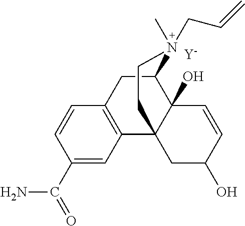 |
| 17 | 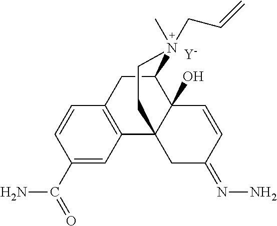 |
| 18 | 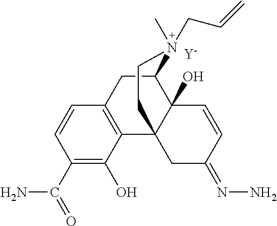 |
| 19 | 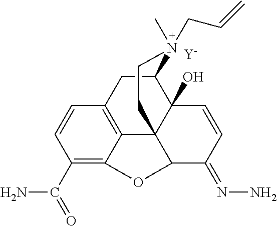 |
| 20 | 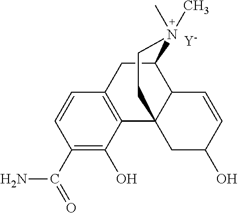 |
| 21 | 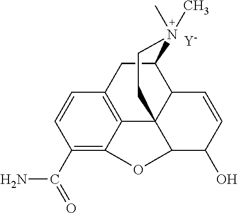 |
| 22 | 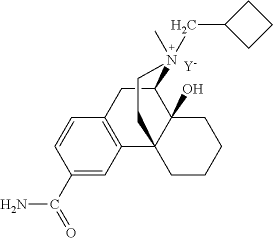 |

In a preferred embodiment, the peripherally-acting μ opioid receptor antagonist is selected from Table A, wherein Y— represents a pharmaceutically acceptable counterion. In a preferred embodiment, Y— is selected from hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate. In a more preferred embodiment, the counterion is selected from bromide, chloride, maleate and malate.

In a more preferred embodiment the peripherally-acting μ opioid receptor antagonist is selected from Compounds 1-4:

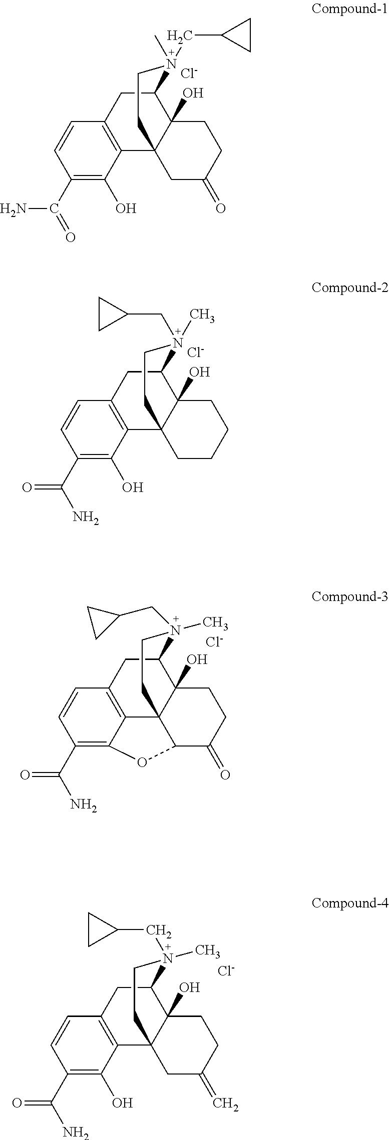

In a more preferred embodiment the peripherally-acting μ opioid receptor antagonist is Compound-1:

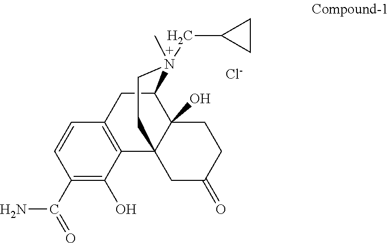

The reduced oral bioavailability as exhibited by the peripherally-acting μ opioid receptor antagonist of the present invention provides improved efficacy for the treatment of gastrointestinal conditions associated opioid analgesic therapy. In addition, the reduction in oral bioavailability does not compromise opiate-induced analgesia in the CNS and surprisingly has a large therapeutic window and is efficacious at lower doses. The decrease in oral bioavailability with improved drug efficacy at lower doses and increased therapeutic window was a significant unexpected improvement.

The peripherally-acting μ opioid receptor antagonists of the present invention are useful in alleviating adverse gastrointestinal conditions associated with opioid analgesic therapy, for example, opioid induced bowel dysfunction, opioid related post operative ileus, constipation, incomplete evacuation, abdominal distention, bloating, abdominal discomfort, and interference with oral drug administration and absorption.

### DEFINITIONS

Listed below are definitions of various terms used to describe this invention. These definitions apply to the terms as they are used throughout this specification and claims, unless otherwise limited in specific instances, either individually or as part of a larger group.

The phrase “condition” refers to a gastrointestinal disease or disorder that develops from the use of the opioid in analgesic therapy. The term “symptom” is any sensation or change in bodily function experienced by a patient that is associated with a particular disease (e.g., a gastrointestinal disease or disorder that develops from the use of opioid analgesic therapy). As used herein, the term “condition” may refer to such disease or disorders such as, for example, opioid induced bowel dysfunction or opioid related post operative ileus.

Opioid induced bowel dysfuction can slow stomach emptying and inhibit bowel movement. The increased time of fecal contents in the intestines results in excessive absorption of water and sodium from fecal contents, resulting in harder, drier stools and constipation.

Constipation is a condition in which a person has uncomfortable or infrequent bowel movements. A person with constipation produces hard stools that can be difficult to pass. The person also can feel as though the rectum has not been completely emptied (incomplete evacuation), suffer from bloating, abdominal distention and discomfort. Acute constipation begins suddenly and noticeably. Chronic constipation, on the other hand, can begin insidiously and persist for months or years. Opioid induced bowel dysfunction afflicts approximately 90% of individuals on analgesic opioids. For chronic pain patients on opioid analgesics, the resulting constipation can lead to frequent dose adjustments affecting a patient's quality of life.

Opioid related post operative ileus is a common problem for patients having undergone surgical procedures, apart from surgery of the abdomen, who suffer from a bowel dysfunction as a result of the opioid analgesia prescribed to control surgical pain. “Ileus,” as used herein, refers to the obstruction of the bowel or gut, especially the colon.

Other symptoms associated with opioid analgesic therapy include but are not limited to dyspepsia, emesis, and interference with oral drug administration and absorption.

The phrase “opioid analgesic” refers to refers to any drug or active agent binds to opioid specific receptors (μ, δ, κ receptors) to alleviate or prevent pain. Opioid analgesics as used herein, include but are not limited, to morphine, diamorphine, fentanyl, alfentanil, buprenorphine, oxycodone, hydromorphone, methadone, codeine, tramadol and butorphanol.

Morphine is a potent opiate analgesic and is derived from the unripe seedpods of the opium poppy (Papaver somniferum). It is commonly used for pain in cancer, myocardial infarction, sickle cell crisis, trauma, kidney stones, severe back pain, palliative care (relieving pain without curing underlying cause) and pain associated with surgical conditions. It is also used as an adjunct to general anesthesia, in epidurial anesthesia, and intrathecal analgesia (painkiller injected into the fluid surrounding the brain and spinal cord). Morphine is also used to treat chronic diarrhea associated with AIDS.

Diamorphine, also known as heroin, is a semi-synthetic opioid drug synthesized from morphine. Diamorphine is prescribed in the United Kingdom for the treatment of acute pain in myocardial infarction, post-surgical pain, and chronic pain caused by cancer. It is given via subcutaneous, intramuscular, intrathecal or intravenous route.

Fentanyl is a strong agonist at the μ-opioid and is approximately 100 times more potent than morphine. Fentanyl is generally prescribed to treat post-surgical pain, chronic pain, and breakthrough pain (acute pain on top of persistent background pain). Fentanyl is administered via an intravenous route, transdermal patch and as a lozenge.

Alfentanil is an analogue of Fentanyl. It has only 1/10th of the potency of fentanyl and only lasts for about ⅓ of the time. However, it starts working four times faster than fentanyl. It is sometimes used for patients who cannot tolerate morphine, diamorphine or oxycodone due to persistent side effects.

Buprenorphine is a semi-synthetic opioid that is used to treat opioid addiction in higher dosages (>2 mg) and to control moderate pain in non-opioid tolerant individuals in lower dosages (˜200 μg). The transdermal formulation is commonly used for chronic cancer pain, musculoskeletal pain (muscles, tendons and ligaments along with the bones), and neuropathic pain (chronic pain resulting from injury to the nervous system).

Oxycodone is an opioid analgesic synthesized from opium-derived thebaine. Oxycodone can be an alternative to morphine for cancer pain. Oxycodone oral medications are generally prescribed to treat pain in diabetic neuropathy, postherpetic neuralgia, osteoarthritis, ambulatory laparoscopic tubal ligation surgery, unilateral total knee arthroplasty, and abdominal gynaecological surgery.

Hydromorphone is a derivative of morphine and is a potent centrally-acting analgesic drug of the opioid class. Hydromorphone is thought to be 3-4 times stronger than morphine.

Hydromorphone is used to relieve moderate to severe pain, and is well known for treating painful, dry cough. In many cases it is preferred over morphine because of its superior solubility, rapid onset, milder side-effects, and lower dependence risk.

Methadone is a synthetic opioid, used medically as an analgesic and as maintenance therapy for use in patients on opioids. Methadone is also used in managing chronic pain owing to its long duration of action and very low cost. Methadone is available in traditional pill, sublingual tablet, and two different formulations designed for the patient to drink.

Codeine is an opiate used for its analgesic, antitussive, and antidiarrheal properties. Codeine is administered via subcutaneous or intramuscular injection, suppositories, as a time released tablet and in cough syrups.

Tramadol is a centrally acting opioid analgesic, used in treating moderate to severe pain and most types of neuralgia (pain along the course of a nerve), including trigeminal neuralgia (inflammation of the trigeminal nerve, causing intense lightning pain in the lips, eye, nose, scalp, forehead, gums, cheek and chin). It is also used off-label for diabetic neuropathy, postherpetic neuralgia (long term pain linked to shingles), fibromyalgia, restless leg syndrome, opiate withdrawal management, migraine, OCD (obsessive-compulsive disorder), and premature ejaculation. Tramadol is available for both injection (intravenous and/or intramuscular) and oral administration.

Butorphanol is a morphinan-type synthetic opioid analgesic. Butorphanol is prescribed for the management of migraine using the intranasal spray formulation. It may also be used parenterally for management of moderate-to-severe pain, as a supplement for balanced general anesthesia, and management of pain during labor. Butorphanol is more effective in reducing pain in women than in men.

The term “agonist,” as used herein, refers to a compound that increases the activity of a receptor. An agonist may either directly interact (e.g., bind) with a receptor or indirectly increase its activity (e.g., to increase the availability of the endogenous neurotransmitter). An agonist refers to a compound which triggers a response by virtue of its interaction (direct or indirect) with a receptor. A “partial agonist” activates a receptor but does not cause as much of a physiological change as does a full agonist.

The term “antagonist” as used herein refers to a compound that decreases the activity of a receptor. An antagonist may either directly interact (e.g., bind) with a receptor or indirectly decrease its activity (e.g., to reduce the availability of the endogenous neurotransmitter). An antagonist also includes compounds which not only fail to activate the receptor with which they interact (directly or indirectly) but also block the receptor's activation by agonists.

The term “peripheral” or “peripherally” designates that the compounds of the present invention acts primarily on physiological systems and components external to the central nervous system. In preferred form, the peripheral opioid antagonists employed in the methods of the present invention exhibit high levels of activity with respect to peripheral tissues, such as gastrointestinal tissue, while exhibiting reduced, and preferably substantially no, central nervous system (CNS) activity.

As used herein, the term “oral bioavailability” is used to describe the fraction of an administered dose of unchanged drug that reaches the systemic circulation. By definition, when a medication is administered intravenously, its bioavailability is 100%. However, when a medication is administered via other routes (such as orally), its bioavailability decreases (due to incomplete absorption and first-pass metabolism) or may vary from patient to patient (due to inter-individual variation). The peripherally-acting μ opioid receptor antagonists of the present invention elicit its primary action locally in the GI tract based on limited systemic exposure following oral administration.

The term “aliphatic group” or “aliphatic” refers to a non-aromatic moiety that may be saturated (e.g. single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆) alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include branched, straight chain and cyclic, substituted or unsubstituted saturated aliphatic hydrocarbon radicals/groups having the specified number of carbons. Preferred alkyl groups comprise about 1 to about 24 carbon atoms (“C₁-C₂₄”) preferably about 7 to about 24 carbon atoms (“C₇-C₂₄”), preferably about 8 to about 24 carbon atoms (“C₈-C₂₄”), preferably about 9 to about 24 carbon atoms (“C₉-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkynyl radicals are “lower alkynyl” radicals having two to about ten carbon atoms such as propargyl, 1-propynyl, 2-propynyl, 1-butyne, 2-butyryl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The terms “halogen” or “halo” as used herein, refers to an atom selected from fluorine, chlorine, bromine and iodine.

The term carbocyclic biaryl refers to fused bicyclic moieties, typically containing 4-20 carbon atoms. An example is naphthalene. The biaryl groups may contain 1-4 heteroatoms. Examples include indoles, isoindoles, quinolines, isoquinolines, benzofurans, isobenzofurans, benzothiophenes, benzo[c]thiophenes, benzimidazoles, purines, indazoles, benzoxazole, benzisoxazole, benzothiazole, quinoxalines, quinazolines, cinnolines, and the like.

The terms “compound” and “drug” as used herein all include pharmaceutically acceptable salts, co-crystals, solvates, hydrates, polymorphs, enantiomers, diastereoisomers, racemates and the like of the compounds and drugs having the formulas as set forth herein.

Substituents indicated as attached through variable points of attachments can be attached to any available position on the ring structure.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described herein, or by resolving the racemic mixtures. The resolution can be carried out in the presence of a resolving agent, by chromatography or by repeated crystallization or by some combination of these techniques, which are known to those skilled in the art. Further details regarding resolutions can be found in Jacques, et al., Enantiomers, Racemates, and Resolutions (John Wiley & Sons, 1981). When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

As used herein, and as would be understood by the person of skill in the art, the recitation of “a compound”—unless expressly further limited—is intended to include salts, solvates, esters, prodrugs and inclusion complexes of that compound.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art.

Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts e.g., salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, carbonate, citrate, cyclopentanepropionate, digluconate, dodecylsulfate, ethanesulfonate, ethanedisulfonate, ethylenediaminetetraacetate (edetate), formate, fumarate, glucoheptonate, glutamate, glycerophosphate, gluconate, hemisulfate, heptanoate, hexanoate, hydroiodide, 2-hydroxy-ethanesulfonate, hydroxynaphthoate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, polygalacturonate, propionate, salicylate, stearate, succinate, sulfate, tannate, tartrate, teoclate, thiocyanate, p-toluenesulfonate, undecanoate, valerate salts, and the like. Representative alkali or alkaline earth metal salts include sodium, lithium, potassium, calcium, magnesium, aluminum, zinc and the like. As used herein, the term “pharmaceutically acceptable ester” refers to esters of the compounds formed by the process of the present invention which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates. Further pharmaceutically acceptable salts include, when appropriate, nontoxic ammonium cations and carboxylate, sulfonate and phosphonate anions attached to alkyl having from 1 to 20 carbon atoms.

The term “pharmaceutically acceptable prodrugs” as used herein refers to those prodrugs of the compounds formed by the process of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound, which is convertible in vivo by metabolic means (e.g. by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191(1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society, (1975); and Bernard Testa & Joachim Mayer, “Hydrolysis In Drug And Prodrug Metabolism: Chemistry, Biochemistry And Enzymology,” John Wiley and Sons, Ltd. (2002).

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such solvents are well known to those skilled in the art, and individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The terms “protogenic organic solvent” or “protic solvent” as used herein, refer to a solvent that tends to provide protons, such as an alcohol, for example, methanol, ethanol, propanol, isopropanol, butanol, t-butanol, and the like. Such solvents are well known to those skilled in the art, and individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. Additionally, the various synthetic steps may be performed in an alternate sequence or order to give the desired compounds. In addition, the solvents, temperatures, reaction durations, etc. delineated herein are for purposes of illustration only and variation of the reaction conditions can produce the desired bridged macrocyclic products of the present invention. Synthetic chemistry transformations and protecting group methodologies (protection and deprotection) useful in synthesizing the compounds described herein include, for example, those described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995).

### Pharmaceutical Compositions

The peripherally-acting μ opioid receptor antagonists of the present invention comprise a therapeutically effective amount of a peripherally-acting μ opioid receptor antagonist of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides).

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The peripherally-acting μ opioid receptor antagonists can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Preferred suitable daily oral dosages for the compounds of the inventions described herein are on the order of about 1 mg to about 300 mg, preferably about 3, 5, 10, 15, 20, 25, 30, 35, 40, 45, 50, 55, 60, 65, 70, 75, 80, 90, 95, 100, 105, 110, 115, 120, 125, 130, 135, 140, 145 to about 150 mg/day, more preferably about 10 to about 100 mg/day, even more preferably about 10 to about 50 mg/day. Dosing schedules may be adjusted to provide the optimal therapeutic response. For example, administration can be one to three times daily for a time course of one day to several days, weeks, months, and even years, and may even be for the life of the patient. Practically speaking, a unit dose of any given composition of the invention or active agent can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth. Unit dose preparations can contain a compound of Formula I or II in the range of about 1 to about 300 mg. Preferably, a unit dose form can contain about 5 to about 150 mg of a compound of Formula I or II, while more preferably a unit dose can have about 5 to about 100 mg of a compound of Formula I or II, while even more preferably a unit dose can have about 5 to about 50 mg of a compound of Formula I or II. In a more preferred embodiment, the invention provides a unit dose of about 5 to 50 mg of Compound-1.

The methods of the present invention can further comprise co-administering peripherally-acting μ opioid receptor antagonists with a therapeutically effective amount of an opioid analgesic, or any combination thereof. Suitable opioid analgesics include but are not limited to, morphine, diamorphine, fentanyl, alfentanil, buprenorphine, oxycodone, hydromorphone, methadone', codeine, tramadol and butorphanol or any combination thereof.

Pharmaceutical kits useful in for example, the treatment of opioid induced constipation which comprise a therapeutically effective amount of an opioid analgesic along with a therapeutically effective amount of the compound of formula I of the invention, in one or more sterile containers, are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL® two-part container (available from Abbott Labs, Chicago, Ill.), as desired. The opioid compound and the compound of Formula I and II may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

Unless otherwise defined, all technical and scientific terms used herein are accorded the meaning commonly known to one with ordinary skill in the art. All publications, patents, published patent applications, and other references mentioned herein are hereby incorporated by reference in their entirety.

### Synthetic Methods

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

The morphinan compounds according to the present invention may be synthesized employing methods taught, for example, in U.S. Pat. No. 5,250,542, U.S. Pat. No. 5,434,171, U.S. Pat. No. 5,159,081, U.S. Pat. No. 4,176,186 U.S. Pat. No. 6,365,594, U.S. Pat. No. 6,784,187 and U.S. Pat. No. 5,270,328. Synthetic methodology for indolylmorphinans is described in Jones et. al. Journal of Medicinal Chemistry, 1998, 41, 4911. Synthetic methodology for pyridomorphinans is described in Ananthan et al, Bioorganic & Medicinal Chemistry Letters, 13, 2003, 529-532. Quaternary morphinan compounds of the instant application can be prepared by methods disclosed in Wentland et. al., PCT/US2008/072632.

### EXAMPLES

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Example 1

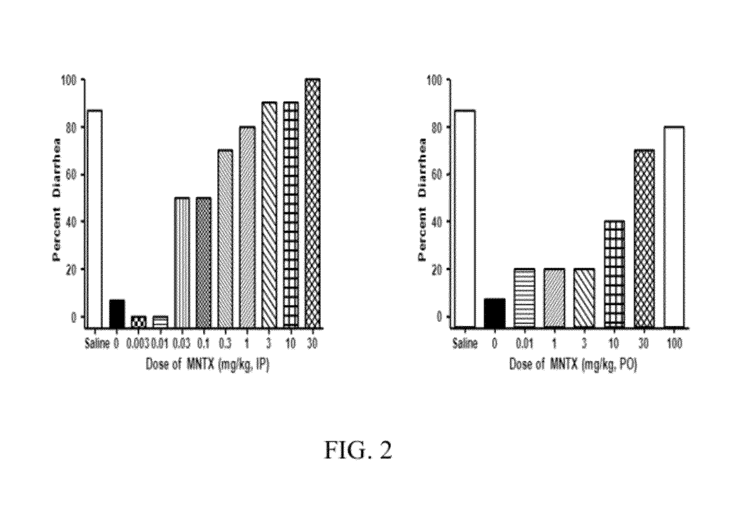

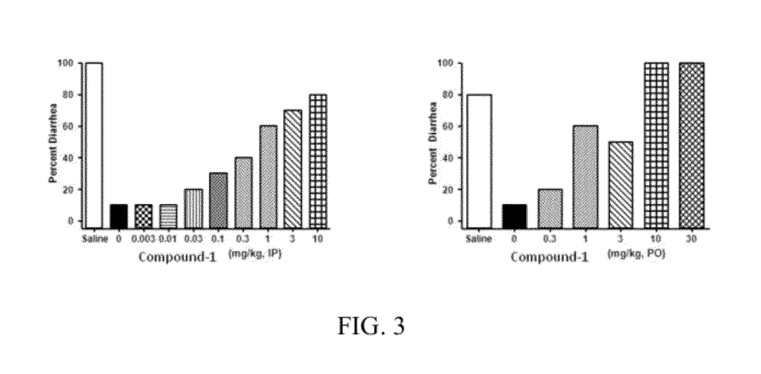

A rodent model of gut motility model (Russell, et al., 1982; Eur. J. Pharm., 78: 255-61) was used to evaluate the bioavailability and efficacy of Compound-1 in rats. The PGE₂ test of gut motility evaluates the inhibition of morphine's side effects. PGE₂ (a prostaglandin) induces diarrhea in mice within 15 minutes of an intraperitoneal injection (0.1 mg/kg). Morphine (1 mg/kg, IP) blocks this effect. Peripherally-acting opioid antagonists inhibit morphine's blockade of PGE₂-induced diarrhea. Male Swiss-Webster mice (n=10/group) were administered Compound-1 or a reference compound 15 minutes (IP or PO) prior to morphine and placed in a Plexiglas pie cage. PGE₂ was administered 30 minutes after morphine. Observations of diarrhea were made 15 minutes after PGE₂ administration. Duration of action was tested by adjusting the time between Compound-1 and morphine administration. FIGS. 2 and 3 show the effectiveness of reference compound methylnaltrexone (MNTX) and Compounds 1-4 to inhibit opioid-induced delay in gut motility by oral and IP administration.

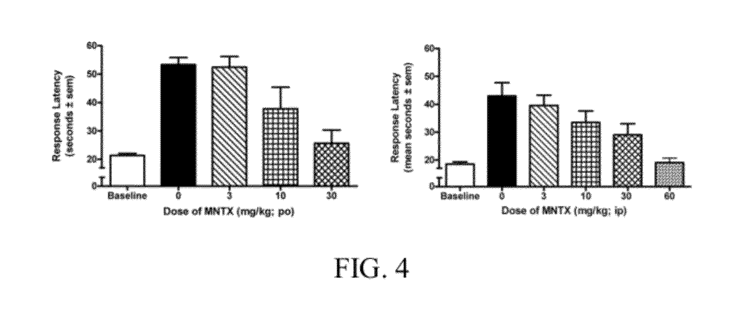

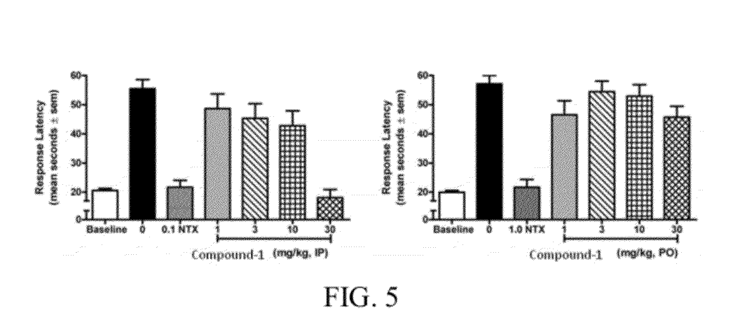

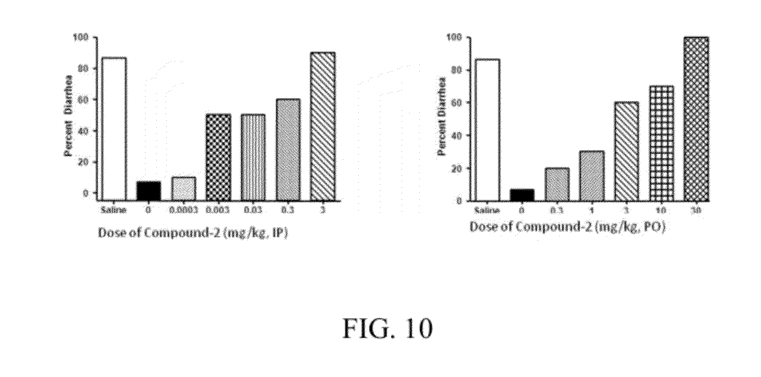

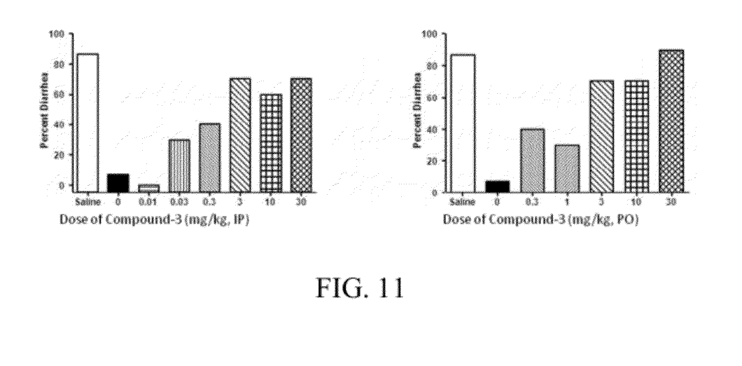

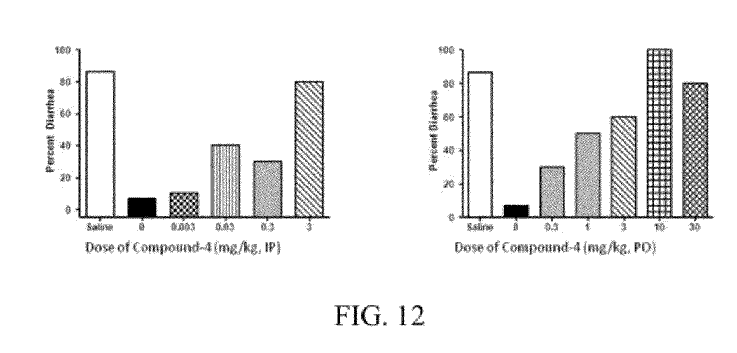

FIG. 4 shows the results of methyl naltrexone (MNTX) in the PGE₂-induced gut motility assay after oral (PO) or intraperitoneal (IP) administration. FIG. 5 shows the results of Compound-1 in the PGE₂-induced gut motility assay after oral (PO) or intraperitoneal (IP) administration. (NTX represents naltrexone). FIG. 10 shows the results of Compound-2 in the PGE₂-induced gut motility assay after oral (PO) or intraperitoneal (IP) administration. FIG. 11 shows the results of Compound-3 in the PGE₂-induced gut motility assay after oral (PO) or intraperitoneal (IP) administration. FIG. 12 shows the results of Compound-4 in the PGE₂-induced gut motility assay after oral (PO) or intraperitoneal (IP) administration.

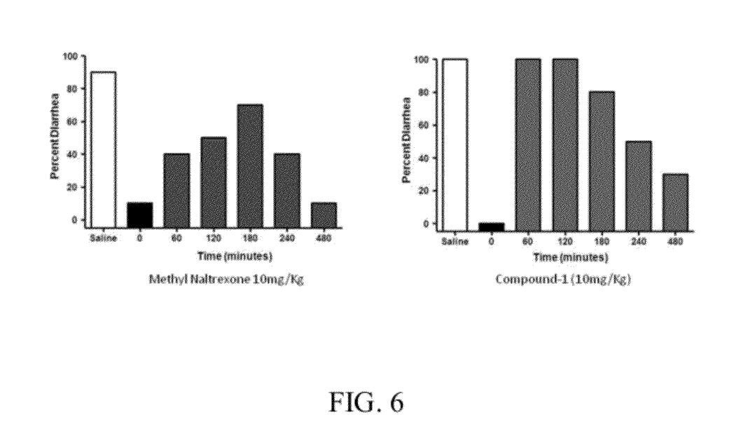

FIG. 6 shows a comparison of the duration of action of oral administration of Compound-1 and methylnaltrexone. The results demonstrate that Compound-1 blocks morphine's effects for up to 4 hours at a dose 3-fold lower than methylnaltrexone.

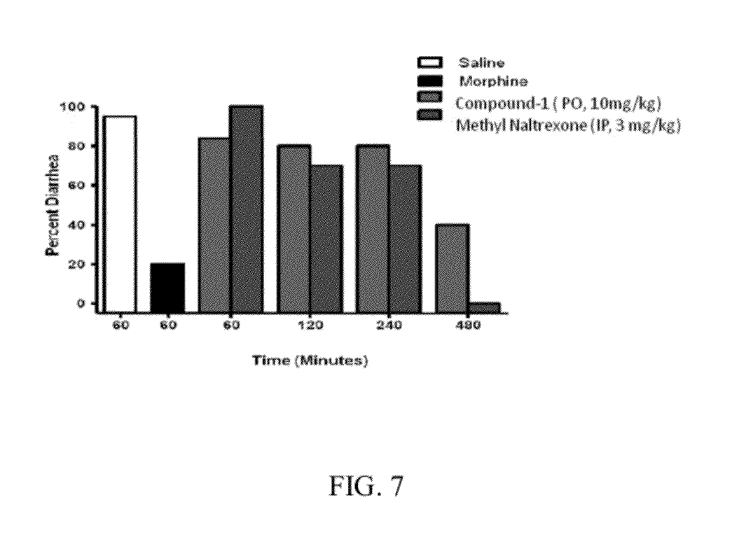

FIG. 7 shows a comparison of the duration of action of the minimal effective IP dose for MNTX and minimal effective PO dose for Compound-1. The results demonstrate that PO administration of Compound-1 has a longer duration of action than MNTX.

### Example 2

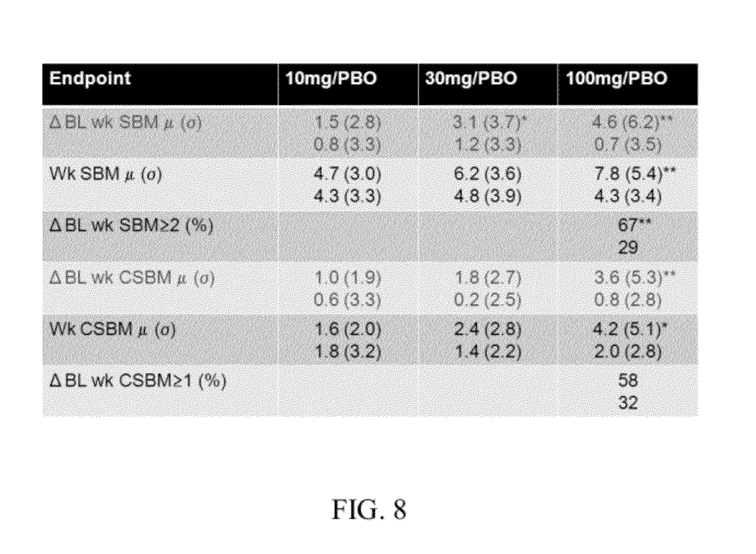

In this multicenter, multi-dose study, 87 patients diagnosed with opioid-induced bowel dysfunction (OBD) during treatment with opioids for chronic, non-cancer pain were randomized to receive escalating doses of Compound-1 (1-100 mg), or placebo with a pre-defined dose escalation schedule. There was a clear dose response with both the 30 mg dose and the 100 mg dose of Compound-1 demonstrating a statistically significant increase in the pre-specified primary endpoint of change from baseline in the number of average spontaneous bowel movements (SBMs), in comparison to placebo. Patients receiving 100 mg Compound-1 once-daily had a mean change from baseline in the average number of SBMs per week of 4.6 versus 0.7 in the placebo group (p=0.003), or a net increase of 3.9 SBMs over placebo. (FIG. 8).

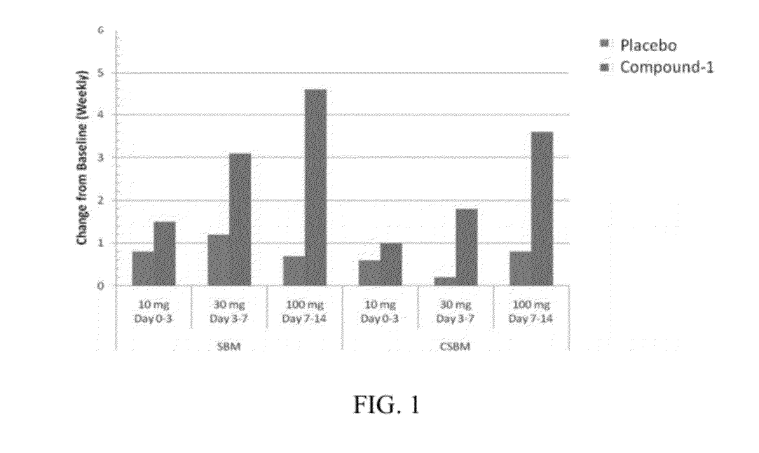

The study also demonstrated a clinically meaningful and statistically significant increase in the average number of complete spontaneous bowel movements (CSBMs) per week from baseline at the 100 mg dose as compared to placebo. The mean change from baseline in CSBMs per week for patients receiving 100 mg Compound-1 was 3.6 versus 0.8 in the placebo group (p=0.006), or a net increase of 2.8 CSBMs over placebo. Importantly, there was no reversal of analgesia as measured by a change in Numerical Pain Rating Scale (NPRS) scores and no increase in opioid use (FIGS. 1 and 8).

### Example 3

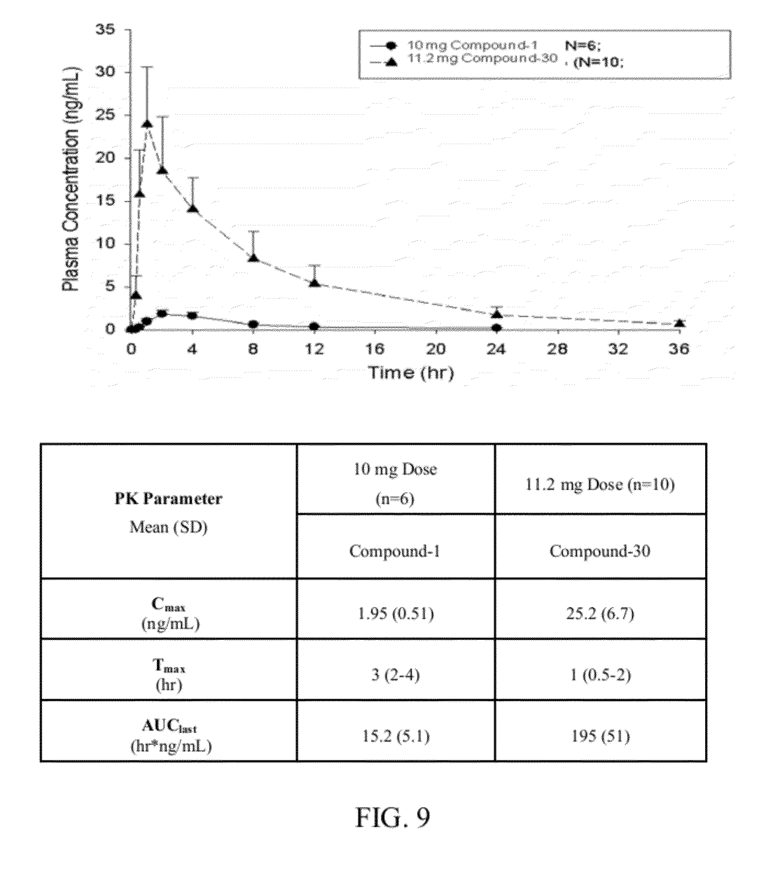

Human PK studies:Pharmacokinetic (PK) evaluation of Compound 1 and Compound-30. To investigate blood concentrations of Compound 1 following oral administration, the absorption and clearance was studied in humans (FIG. 9).

The structure of Compound-30 is given below:

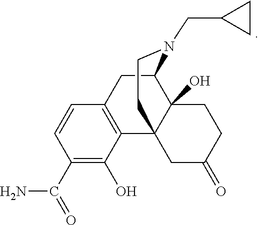

While this invention has been particularly shown and described with references to preferred embodiments thereof, it will be understood by those skilled in the art that various changes in form and details may be made therein without departing from the scope of the invention encompassed by the appended claims.

1. A method of alleviating the adverse conditions associated with opioid analgesic therapy by the oral administration of a compound of Formula I: 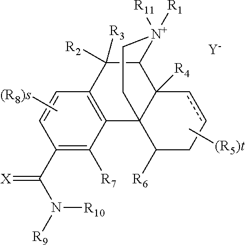
wherein:
s is 0, 1 or 2;
t is 0, 1, 2, 3, 4, 5, 6, or 7;
Y— is a pharmaceutically acceptable counterion;
X is S or O;
each R₂, R₃, R₄, R₅, R₆, R₇ and R₈ is independently selected from absent, hydrogen, halogen, —OR₂₀, —SR₂₀, —NR₂OR₂₁, —C(O)R₂₀, —C(O)OR₂₀, —C(O)NR₂₀R₂₁, —N(R₂₀)C(O)R₂₁, —CF₃, —CN, —NO₂, —N₃, acyl, alkoxy, substituted alkoxy, alkylamino, substituted alkylamino, dialkylamino, substituted dialkylamino, substituted or unsubstituted alkylthio, substituted or unsubstituted alkylsulfonyl, optionally substituted aliphatic, optionally substituted aryl, heterocyclyl or substituted heterocyclyl; or alternatively, two of R₂, R₃, R₄, R₅, R₆, R₇ and R₈ together with the atoms they are attached to form an optionally substituted ring; alternatively R₂ and R₃ together with the carbon they are attached to form a C═X group; wherein each R₂₀ and R₂₁ is independently selected from absent, hydrogen, halogen, —OH, —SH, —NH₂, —CF₃, —CN, —NO₂, —N₃, —C(O)OH, —C(O)NH₂, acyl, alkoxy, substituted alkoxy, alkylamino, substituted alkylamino, dialkylamino, substituted dialkylamino, substituted or unsubstituted alkylthio, substituted or unsubstituted alkylsulfonyl, aliphatic, substituted aliphatic, aryl or substituted aryl; and
each R₁ and R₁₁ is selected from aliphatic, substituted aliphatic, aryl, substituted aryl; alternatively two R₅ groups, or an R₅ and an R₆ group, together with the carbon they are attached to form a C═X group;
each R₉ and R₁₀ is selected from hydrogen, aliphatic, substituted aliphatic, aryl, substituted aryl, heterocyclyl or substituted heterocyclyl;
wherein, said compound of formula I is administered in a daily dosage of about 1 to about 300 mg/day.

2. A method of alleviating the adverse conditions associated with opioid analgesic therapy by the oral administration of a compound of Formula II: 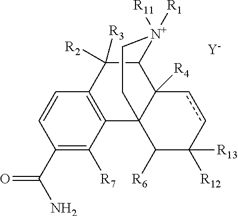
wherein;
t is 0, 1, 2, 3, 4, 5, 6, or 7;
Y— is a pharmaceutically acceptable counterion;
each R₂ and R₃ is independently selected from hydrogen, halogen, —OR₂₀, and —SR₂₀; alternatively R₂ and R₃ together with the carbon they are attached to form a C═X group;
each R₄, R₆, and R₇ is independently selected from absent, hydrogen, halogen, —OR₂₀, —SR₂₀, —NR₂OR₂₁, —C(O)R₂₀, —C(O)OR₂₀, —C(O)NR₂OR₂₁, —N(R₂₀)C(O)R₂₁, —CF₃, —CN, —NO₂, —N₃, acyl, alkoxy, substituted alkoxy, alkylamino, substituted alkylamino, dialkylamino, substituted dialkylamino, substituted or unsubstituted alkylthio, substituted or unsubstituted alkylsulfonyl, aliphatic, substituted aliphatic, aryl, substituted aryl, heterocyclyl or substituted heterocyclyl; alternatively, two of R₆ and R₇ together with the atoms they are attached to form an optionally substituted ring;
each R₁ and R₁₁ is selected from aliphatic, substituted aliphatic, aryl, substituted aryl, heterocyclyl or substituted heterocyclyl.

3. The method according to claim 1, wherein said compound is selected from Table A:

| | |
|---|---|
| 1 | 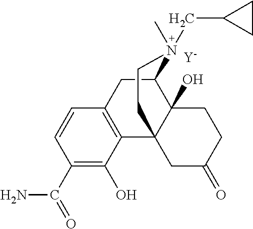 |
| 2 | 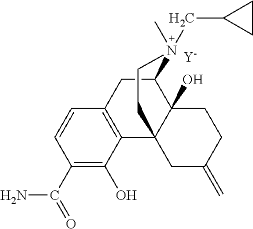 |
| 3 | 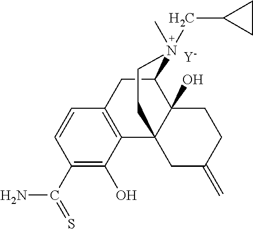 |
| 4 | 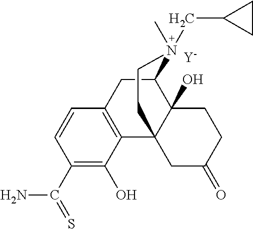 |
| 5 | 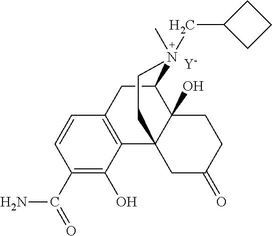 |
| 6 | 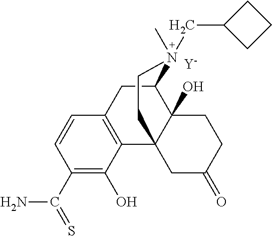 |
| 7 | 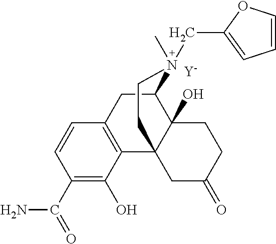 |
| 8 | 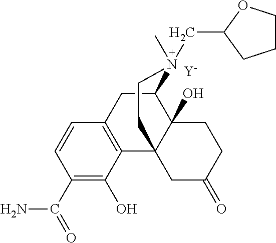 |
| 9 | 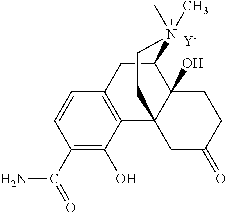 |
| 10 | 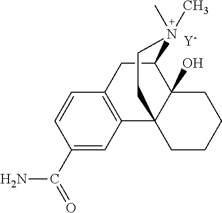 |
| 11 | 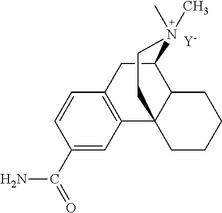 |
| 12 | 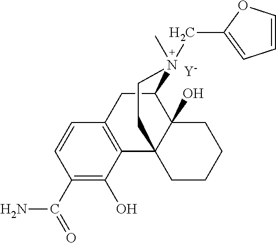 |
| 13 | 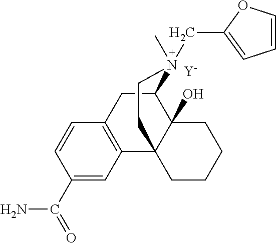 |
| 14 | 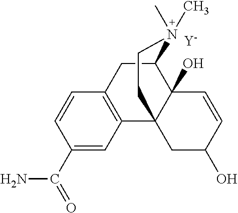 |
| 15 | 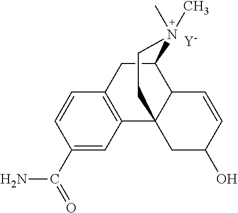 |
| 16 | 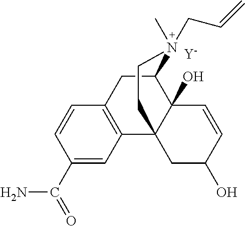 |
| 17 | 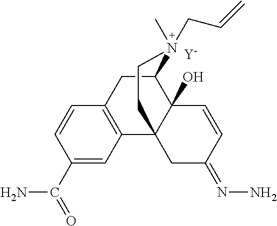 |
| 18 | 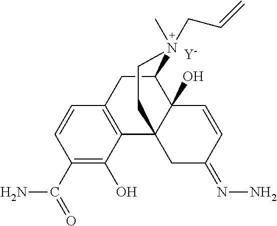 |
| 19 | 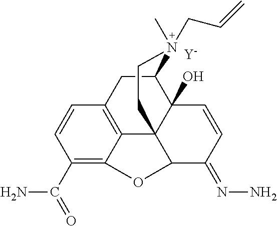 |
| 20 | 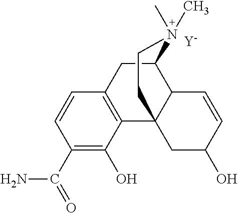 |
| 21 | 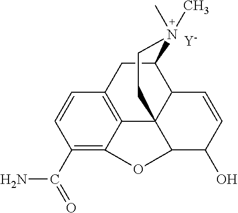 |
| 22 | 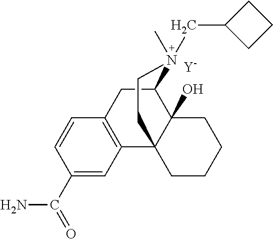 |

4. The method according to claim 1, wherein Y— is selected from hydroxide, acetate, benzenesulfonate (besylate), benzoate, bicarbonate, bisulfate, carbonate, camphorsulfonate, citrate, ethanesulfonate, fumarate, gluconate, glutamate, glycolate, bromide, chloride, isethionate, lactate, maleate, malate, mandelate, methanesulfonate, mucate, nitrate, pamoate, pantothenate, phosphate, succinate, sulfate, tartrate, trifluoroacetate, p-toluenesulfonate, acetamidobenzoate, adipate, alginate, aminosalicylate, anhydromethylenecitrate, ascorbate, aspartate, calcium edetate, camphorate, camsylate, caprate, caproate, caprylate, cinnamate, cyclamate, dichloroacetate, edetate (EDTA), edisylate, embonate, estolate, esylate, fluoride, formate, gentisate, gluceptate, glucuronate, glycerophosphate, glycolate, glycollylarsanilate, hexylresorcinate, hippurate, hydroxynaphthoate, iodide, lactobionate, malonate, mesylate, napadisylate, napsylate, nicotinate, oleate, orotate, oxalate, oxoglutarate, palmitate, pectinate, pectinate polymer, phenylethylbarbiturate, picrate, pidolate, propionate, rhodanide, salicylate, sebacate, stearate, tannate, theoclate, tosylate.

5. The method according to claim 1, wherein Y— is bromide, chloride, maleate and malate.

6. A method according to claim 1, wherein the condition is a disease or disorder or a symptom of said disease or disorder associated with opioid analgesic therapy.

7. A method according to claim 1, wherein the condition is associated with the gastrointestinal system.

8. A method according to claim 7, wherein said gastrointestinal condition comprises opioid induced bowel dysfunction, opioid related post operative ileus, constipation, incomplete evacuation, abdominal distention, bloating, abdominal discomfort, and interference with oral drug administration and absorption.

9. A method according to claim 8, wherein the gastrointestinal condition is opioid-induced bowel dysfunction.

10. A method according to claim 8, wherein the gastrointestinal condition is opioid related post operative ileus.

11. The method according to claim 1, wherein the opioid analgesic therapy is selected from the group consisting of morphine, diamorphine, fentanyl, alfentanil, buprenorphine, oxycodone, hydromorphone, methadone, codeine, tramadol and butorphanol.

12. A method according to claim 1, wherein said compound is administered in a daily dose of about 5 to about 150 mg/day.

13. A method according to claim 1, wherein said compound is administered in a daily dose of about 5 to about 100 mg/day.

14. A method according to claim 1, wherein said compound is administered in a daily dose of about 5 to about 50 mg/day.

15. A method of treating or preventing pain while inhibiting opioid-induced bowel dysfunction comprising oral administration to a patient in need of such treatment, a therapeutically effective amount of compound according to claim 1.

16. A unit dosage formulation for alleviating the adverse conditions associated with opioid analgesic therapy by the oral administration of a compound according to claim 1 wherein said unit dosage form is between about 5 and 100 mg/day.

17. The unit dosage formulation of claim 16, wherein said formulation comprises a compound of Table A in about 5 to about 100 mg/day.

18. The unit dosage formulation of claim 17, wherein said compound of Table A has reduced peripheral opioid activity in comparison with methyl naltrexone of same dose administered in said unit dosage formulation.

19. The method according to claim 1 wherein said compound of Formula I or II is Compound-1:

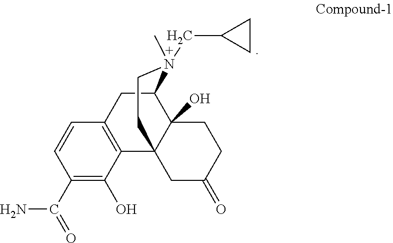

20. The method according to claim 1, wherein said compound of formula is: 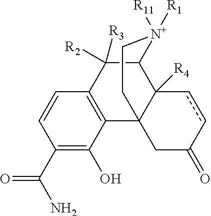
Wherein R₂ and R₃ are hydrogen;
R₄ is selected from hydrogen and hydroxyl;
R₁ and R₁₁ are aliphatic;
wherein, said compound of formula I is administered in a daily dosage of about 1 to about 300 mg/day.
